(19) United States
(12) Patent Application Publication
(10) Pub. No.: US 2024/0374865 A1
(43) Pub. Date: Nov. 14, 2024

(54) MINIMALLY INVASIVE URINARY CATHETER

(71) Applicant: Besancon Innovations, LLC, Victoria, TX (US)

(72) Inventor: Mark Besancon, Victoria, TX (US)

(21) Appl. No.: 17/758,581

(22) PCT Filed: Jan. 25, 2022
(86) PCT No.: PCT/US2022/070334

§ 371 Date: Jul. 11, 2022

(51) Int. Cl. A61M 25/00

(52) U.S. Cl. CPC A61M 25/0017; A61M 25/0043

(57) ABSTRACT

Various embodiments of the systems, methods and devices are provided for improved drainage of a mammalian bladder wherein a urinary drainage catheter is provided that does not extend into, or physically engage, the bladder. This configuration allows for improved drainage, with avoidance of common crystal blockages, as well as improved flushing capabilities of the catheter and portions of the mammalian urethra not covered by the shaft of the drainage catheter.

### CROSS-REFERENCE TO RELATED APPLICATIONS

This application is a national stage application of PCT/US2022/70344, filed Jan. 25, 2022 and titled MINIMALLY INVASIVE URINARY CATHETER, the entire content of which is incorporated herein by reference.

### STATEMENT REGARDING FEDERALLY SPONSORED RESEARCH OR DEVELOPMENT

None

### BACKGROUND OF THE INVENTION

### Field of the Invention

The invention relates to systems, devices and methods for drainage and/or flushing of the mammal bladder.

### Description of the Related Art

A variety of catheters and techniques are known for draining and/or flushing an obstructed animal bladder and/or urethra. Mammal bladders are in fluid communication with the urethra, which is essentially a tube leading to an outflow opening at, e.g., the tip of the penis for male patients. At the point where the urethra and bladder reservoir communicate, a sphincter muscle is provided which serves essentially as a valve that, when closed, prevents urine from flowing into the urethra and, when open, allows urine to drain from the bladder into the urethra and out of the patient's body. This normal physiological response wherein the sphincter muscle controls urine flow may be described as an active draining process.

Urinary catheters that allow port-like access to the urinary bladder of a patient by utilizing the patient's urethra are known. See, e.g., U.S. Pat. No. 10,342,412 and US Publication 20190262579, each teaching a catheter with access through the patient's urethra, wherein a distal portion of the catheter's shaft extends through the bladder sphincter muscle into the patient's bladder.

In addition, so-called Tom Cat or TomCat Catheters, e.g., Argyle™, are known in the veterinary arts for use with male feline patients, wherein the catheter shaft is approximately 11.4 cm in length with a 3.5 Fr outer diameter and 24 gauge inner diameter. However, these devices also require a shaft that extends along the length of the urethra such that a distal portion of the shaft is located within the bladder.

Without limitation, the male urethra, e.g., again without limitation the feline male urethra, comprises a relatively narrowed diameter urethral opening at or near the distal opening at the penis. It is in this region where urinary crystals are prone to collect and cause flow blockage. The remainder of the urethra comprises a larger diameter that functions to naturally facilitate crystal flow therealong, without blockage.

In these known catheters, urine and urinary crystals are allowed to remain in the bladder due to the positioning of the shaft opening with the patient's bladder which often results in more return blockages in the catheter shaft lumen. In addition, such an arrangement often results in bladder wall irritation, longer than necessary recovery times and, in some cases, infections requiring additional treatment. Moreover, as a result of the known catheter shafts occupation of the entire length of the patient's urethra, with the distal end of the catheter shaft located within the patient's bladder, these known devices cannot be easily used to assist in flushing the patient's urethra which may be desirable in certain situations.

In addition, the known urinary catheters are required to pass through the urinary sphincter muscle, effectively rendering that muscle non-functional as it is unable to control urinary flow from the bladder when so engaged with the catheter shaft. In such cases, therefore, the urine is drained in a passive, uncontrolled manner. The catheter shaft also comprises a point of irritation and potential infection at the point of engagement with the urinary sphincter.

Further, it is a general principle that, to the extent possible, retention of natural biological function is preferred when developing medical devices. Stated differently, it is highly preferred to not eliminate natural biological function when solving a problem with a medical device or technique. With this general principle in mind, it would therefore be desirable allow the urinary sphincter muscle to retain its natural function when considering urinary catheterization of at least male felines.

It would be highly desirable to provide a urinary catheter that addresses these issues.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a side cutaway view of a bladder with crystalline blockage at a distal, narrowed, end of the urethra.

FIG. 2 illustrates a prior art device.

FIG. 3 illustrates a side view of one embodiment of the present device disposed in a portion of the urethra.

FIG. 4 illustrates a perspective view of one embodiment of the present device

FIG. 5A illustrates a side cutaway view of one embodiment of the present device.

FIG. 5B illustrates an end view of the embodiment of FIG. 5A.

FIG. 6 illustrates a side view of the embodiment of FIGS. 5A and 5B.

### DETAILED DESCRIPTION OF THE INVENTION

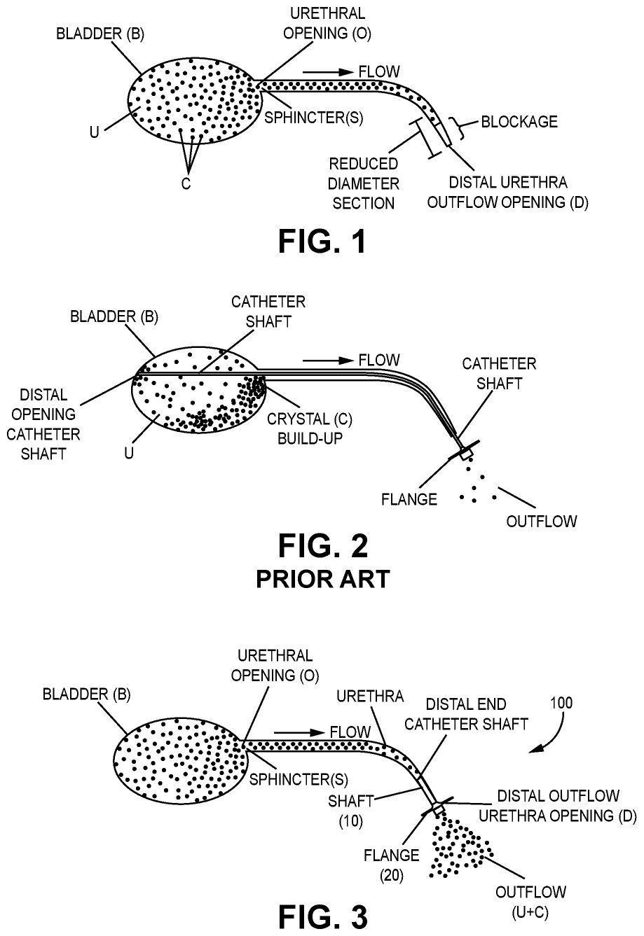

FIG. 1 illustrates the basic mammalian anatomical system comprising a bladder B having an opening O comprising a sphincter muscle S, whereby the bladder B is in controlled fluid communication with the urethra U. The urethra U defines a lumen L that, as shown, may comprise an effectively constant inner diameter until reaching a distal region of narrowed inner diameter. Ultimately, the urethra terminates at a distal opening D, i.e., the tip of the patient's penis, where outflow of urine U normally occurs. Thus, the normal urine flow comprises a filling of the bladder B with urine and urinary crystals C. The sphincter S functions normally as a valve and generally is closed thereby preventing fluid drainage from the bladder B. When the sphincter S opens to enable urination, i.e., draining of the urine U and crystals C from the bladder B, urine U and crystals C flow from the bladder B through the urethra and out through the distal outflow opening D.

FIG. 1, however, illustrates a typical urethral blockage wherein crystals C have aggregated at the narrowed diameter section of the urethra, effectively blocking urine U flow therethrough.

FIG. 2 illustrates a known, prior art solution to the blockage condition of FIG. 1. Here, a catheter having a shaft defining a lumen therethrough is inserted through and along the length of the urethra such that a distal opening of the catheter shaft is positioned inside the bladder B. This requires the catheter shaft to engage and pass through the sphincter muscle S, effectively disabling its valving functionality. In this configuration, urine U flows out of the bladder B in an involuntary, passive and uncontrolled manner through the lumen of the catheter shaft. As discussed supra, the length of these known shafts extend along the full length of the urethra into the bladder B and may comprise lengths of approximately 11.4 cm and inner bore or diameter that are about 24 gauge. Consequently, due to the positioning of the distal end of the catheter shaft within the bladder B, only incomplete drainage is possible as is shown. As the skilled artisan will understand, the distal end of the catheter shaft within the bladder B must be in contact with urine U in order for draining to occur. When the draining urine U level drops such that no further draining occurs, i.e., the distal end and lumen of the catheter shaft are no longer submerged, the rest of the urine U remains in the bladder B resulting in, inter alia, increase in potential for infection and irritation. In addition, as shown in FIG. 2, crystals C are collected at or near the urethral opening of the bladder B, resulting in potential blockage at that point as well as increasing potential for infection and irritation. Finally, the length of the illustrated shaft requires that it either be withdrawn partially or completely to allow a flushing of the urethra and/or bladder to occur B.

Turning now to FIG. 3, an embodiment of the present invention is illustrated. As shown, a urinary drainage catheter 100 comprises a shaft 10 that is only disposed within the narrowed, narrowing or reduced diameter section of the urethra. In some embodiments, the shaft 10 may extend beyond the narrowed diameter section of the urethra, but in no case will the shaft 12 extend to engage the bladder sphincter muscle S at the urethral opening O, or extend into the bladder B itself.

This arrangement wherein the catheter shaft 10 extends along a partial length of the urethra allows for complete drainage of the urine U and crystals C within the bladder, as controlled by the patient's sphincter muscle S at the urethral opening O. Consequently the natural biological urinary function is maintained with no risk of bladder B or sphincter muscle S irritation or infection as the catheter shaft does not contact either of those structures. In addition, crystals C do not collect at the urethral opening O as in FIG. 2 because the natural, sphincter-controlled function works to effectively drain the crystals C into the urethra and through the distally positioned catheter shaft S. In the event that a urethral crystalline blockage does occur, the shortened shaft device 100 of FIG. 3 may be used to effectively flush away the crystals C for subsequent draining and removal. As known in the art, saline or other fluid may be used and flushed through the lumen into the urethra by accessing the lumen 12 at the proximal end 16 of the catheter shaft 10. Because at least some of the urethra is not covered or engaged by the catheter shaft 10, the fluid flush will be effective in those areas.

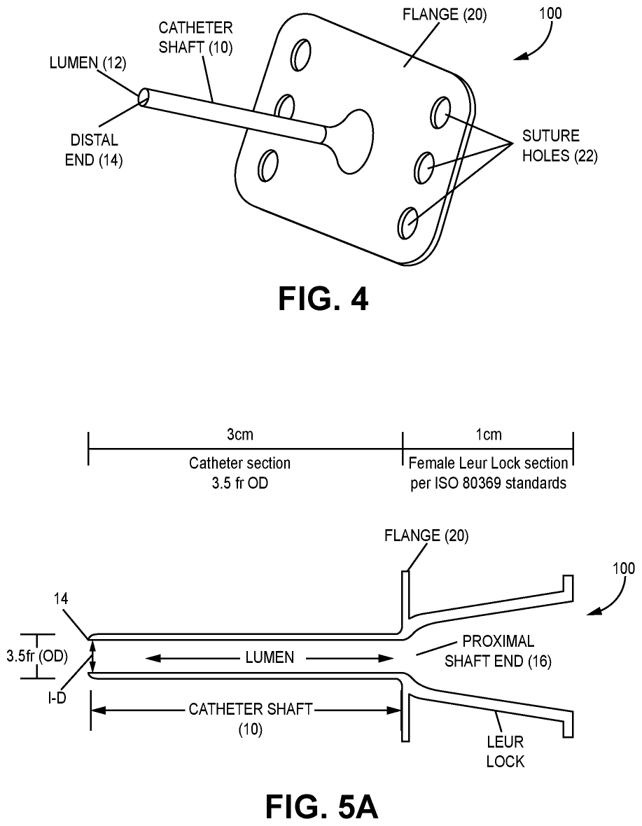

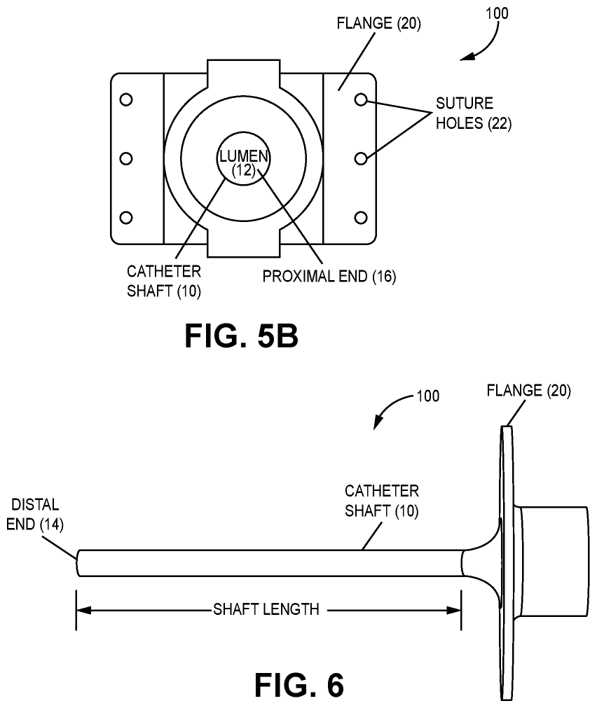

FIG. 4 provides a perspective view of one embodiment of the present invention that may be used as describe in connection with FIG. 3. As shown, the catheter shaft 10 preferably comprises a length of 2-3 cm in an exemplary male feline application, thereby ensuring that no contact will occur with the bladder sphincter muscle S and, therefore, cannot extend into the bladder B. Any length of shaft S may be employed, depending on the distance from the penis tip, or distal urethral outflow opening D), to the mammalian patient's bladder B/urethral opening O and related sphincter muscle S, so long as the shaft 10 extends into the urethra and does not engage the bladder sphincter muscle S or enter the bladder B. For example, again depending on the procedure and the relevant anatomical distance involved, a shaft length of less than 3 cm. measured from the flange 20 (see FIGS. 5A and 5B), or less than 4 cm, or less than 5 cm, or less than 6 cm, among other lengths, may be employed. The proximal end 16 of the shaft 10 is connected with a flange 20 used to facilitate placement and comprising a plurality of suture apertures or holes 22 therethrough. The shaft 10 comprises a lumen 12 having a larger inner diameter I-D than that of the known catheter of FIG. 2. Here, the inner diameter I-D is preferably fixed at 22 gauge, as compared with an inner diameter of 24 gauge in the known device of FIG. 2. The lumen 12 of the shaft 10 extends through the shaft 10 and through the flange 20 to provide a distal opening or end at 16 and a proximal opening or end at 14. The lumen 12 comprises a constant inner diameter I-D therethrough, as discussed this may be fixed at 22 gauge, though other inner diameters I-D may also be used, e.g., 20-24 gauge, without limitation.

Figures 5A and 5B provide, respectively, a cutaway side view and an end view of one embodiment of the urinary drainage catheter 100. Thus, the catheter shaft 10 is shown with the flange 20 and an optional leur lock section at the proximal end of the device 100. Preferably, as discussed. the shaft 10 may have a constant outer diameter of 3.5 Fr and a constant inner diameter I-D of 22 gauge, though these parameters may be modified while still maintaining the inventive objectives described herein. The lumen 12 defined by the shaft 10 extends through the catheter 100 so that fluid flow therethrough is enabled.

FIG. 6 shows a side view of the device of FIGS. 5A and 5B.

The inventive device may comprise a molded, unitary structure or may comprise elements that are separately manufactured, but subsequently connected or attached. The device may comprise a polymer or blend thereof. A preferred material comprises polypropylene, though other materials may also be used as the skilled artisan will understand.

The urinary catheter embodiments described herein may preferably be used to in connection with any mammal to facilitate natural function bladder draining and/or flushing of the urethra. A preferred mammal is the male feline, but the inventions described herein are not so limited.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1.-7. (canceled)

8. A urinary drainage catheter for insertion into a portion of the length of a mammalian urethra for non-passive draining of urine from the animal's bladder, comprising:
a catheter shaft comprising a constant outer diameter, a constant inner diameter and defining a lumen therethrough, the shaft having a distal end and a proximal end; and
a flange disposed at the proximal end of the shaft,
wherein the shaft length from the flange to the distal end of the shaft is configured to extend along only a portion of the length of the urethra.

9. The catheter of claim 8, wherein the distal end of the inserted catheter shaft is positioned at a point that is proximal of the patient's urethral opening.

10. A method for mitigating urinary crystal blockages in a mammalian urethra while maintaining natural sphincter control of bladder drainage, comprising:
providing a urinary drainage catheter comprising:
a catheter shaft comprising a constant outer diameter, a constant inner diameter and defining a lumen therethrough, the shaft having a distal end and a proximal end; and
a flange disposed at the proximal end of the shaft;
ensuring that the length of the catheter shaft distal of the flange is less than the length of the urethra;
inserting the catheter shaft into the urethra; and
allowing normal sphincter controlled bladder drainage to continue.

11. The method of claim 10, wherein a region of the urethra comprises a narrowed diameter section having a length, and ensuring the inserted catheter shaft extends at least along the length of the narrowed diameter section.

12. The method of claim 10, further comprising ensuring that the distal end of the shaft does not extend to engage or extend into the bladder.

13. The method of claim 10, further comprising flushing a fluid into the proximal end of the shaft, thereby flushing the portion of the animal's urethra that is not covered or engaged by the catheter shaft.
